(19) United States
(12) Patent Application Publication
(10) Pub. No.: US 2007/0053852 A1
(43) Pub. Date: Mar. 8, 2007

(54) Lipidic protein-transfer systems and cosmetic/dermatological use thereof

(76) Inventors: Rolf D. Beutler, HOCHST/HUMMETROTH (DE); Karl-Heinz Schmidt, Gomaringen (DE); Eberhard Nurnberg, Uttenreuth/Weiher (DE); Karl-Heinz Schrader, Holzminden (DE)

Correspondence Address: WILLIAM COLLARD;COLLARD & ROE, P.C., 1077 NORTHERN BOULEVARD, ROSLYN, NY, 11576, US

(21) Appl. No.: 10/558,310

(22) PCT Filed: Dec. 19, 2003
(86) PCT No.: PCT/EP03/14648

§ 371 Date: Nov. 28, 2005

(30) Foreign Application Priority Data
May 28, 2003 (DE) 103 24 256.2

(51) Int. Cl. A61K 8/73

(52) U.S. Cl. 424/59; 424/70.12; 424/70.13

(57) ABSTRACT

The present invention relates to compositions having bioactive transfer proteins in stable carrier systems. Such systems are above all suitable for influencing/changing lipid cell structures by means of improved lipid exchange, while at the same time having great stability. In this connection, not only transfer proteins and lipid components, but also protective colloids are used, by means of which an impairment of the transfer effect of the proteins and a loss of stability can be avoided. The compositions are suitable for external topical use for cleansing, care, dermatological and/or therapeutic treatment of the skin, hair, or mucous membranes.

### OBJECT OF THE INVENTION

The present invention relates to compositions having bioactive lipid transfer proteins in stable carrier systems. Such systems are particularly suitable for influencing/changing lipid cell structures by means of improved lipid exchange, while at the same time having great stability. In this connection, not only transfer proteins and lipid components, but also, in particular, protective colloids are used, by means of which an impairment of the transfer effect of the proteins and a loss of stability can be avoided. The compositions are particularly suitable for external topical use, for cleansing, care, dermatological and/or therapeutic treatment of the skin, hair, or mucous membranes.

### BACKGROUND OF THE INVENTION

Active substance systems for lipid exchange from and to target structures are known from DE-PS 3815 473 and the parallel patents FR 2,631,236, U.S. Pat. No. 5,776,470, U.S. Pat. No. 6,077,520. These are characterized in that at least one amphiphilic and/or lipophilic component having at least one water-soluble transfer protein forms the active substance system. In general, emulsions or creams are mentioned as bases for application to the skin. However, no special formulations are disclosed.

Also, lipoprotein creams in the form of O/W emulsions are known, which contain at least one polar oil as the oil component, and a plant protein as an emulsifier (e.g. from 3 to 10 wt.-%), see EP-B-1 047 396. Preferred emulsifiers are lipoproteins from oats, wheat, or peas. The oil components (1 to 20%) must have specific surface tension values, and are therefore preferably selected from among C₂-C₆ alkanols or the C₈-C₂₂ alkanol esters of monovalent or multivalent C₂-C₆ hydroxyalkanols or the C₈-C₂₂ alkanol esters of monovalent or multivalent C₂-C₆ hydrocarboxylic acids. Ionic and hydrophilic O/W emulsifiers having an HLB value of 5 or more should not be used in such formulations. Usual galenic aids such as thickeners (plant gums, cellulose ether, synthetic or natural polymers), as well as co-emulsifiers such as cetyl/stearyl alcohol (0.1 to 1 part by weight per part by weight of oil component), active substances and care substances can be contained as additives.

Products that can be applied topically and contain traditional hydrophilic O/W emulsifiers are known from EP-A-0 532 465. There, very specific leguminose fractions (soybean, peas, not grains) are used for skin care, particularly for the treatment of inflammatory skin diseases.

According to the European patent EP-B-0 835 674, creams that can be obtained as a foaming O/W emulsion when dispensed from suitable devices are produced from a fatty phase, water, and 0.1 to 2% of a gel-forming system of crosslinked acrylate homopolymers or crosslinked acrylate copolymers. In this connection, maximally 2% of usual O/W emulsifiers such as sodium lauryl sulfate are supposed to be contained. Acrylate/acrylate copolymers such as Pemulen™ are suitable as emulsifying polymers.

Such hydrodispersion gels are described in general in an overview by R. Daniels, Pharm. Ztg. 147, (Oct. 24, 2002). In contrast to the “usual” emulsions, in the case of these products, the required stabilization does not take place with so-called traditional emulsifiers, as mentioned, but rather using suitable macromolecules, i.e. using gel forming agents (see above, EP-B-0835 674). The polymer additive results in a thickening effect and imparts a flow limit to the outer phase. Such systems are also referred to as quasi-emulsions. The stated hydrodispersion gels contain carbomer, for example, as the polymer, and a silicone oil in the form of phenyl trimethicone, or also capryl/caprinic acid triglycerides or other oils. Here, the absence of more polar substances such as partial esters of multivalent alcohols or higher fatty alcohols is characteristic for the systems described in the publication cited above. Likewise, no products that contain protein are described as ingredients.

DE 100 61 419 relates to the use of protein hydrolysates as an active substance for restructuring keratinous fibers of hair.

In CH B 686 554, cosmetic agents from protein hydrolysates without further characterization and water/glycerin are described. Such agents are worked into cosmetic products in conventional manner.

In the U.S. patent application US A1 2002/0054918, agents containing pharmaceutical hydrogen peroxide for the treatment of inflamed skin areas are described. In this connection, moisturizing agents such as hyaluronic acid, wheat protein without further characterization, glycerin or dimethicone, tocopherol can additionally be used, if necessary, e.g. in the production of lotions, creams, emulsions, or ointments, in conventional manner.

WO 00/64472 describes compositions for the treatment of dermatological problems, containing fruit extracts such as pomegranate extract, in order to neutralize free radicals. Known bases such as starch, polyvinyl pyrrolidone and the like are described as carriers for the fruit extract. In addition, moisturizing agents such as hydroxy polycarboxylic acids, dimethicone, glycerin, sodium chloride, or wheat protein without special characterization are used as carriers for the fruit extract.

In total, this shows that protein hydrolysates or proteins have been used until now as active substances, particularly moisturizers, in cosmetic products for improving skin or hair. In this connection, conventional cosmetic formulations were produced, particularly using conventional emulsifiers, particularly O/W emulsifiers.

### TASK OF THE INVENTION

In view of the undesirable properties of emulsifiers, particularly emulsifiers of the traditional O/W type, such as ionic substances, such as, for example, sodium lauryl sulfate or the like, or also non-ionic O/W emulsifiers of the type of macrogol derivatives with higher alcohols or alcohol fatty acids, compositions are supposed to be made available, particularly for products that can be applied externally, i.e. topically, which are stable despite the absence of such traditional emulsifiers, and whereby proteins can be used as lipid transfer components, without their effectiveness being impaired, for example as the result of an emulsifying effect.

### EXPLANATION OF THE INVENTION

This task is accomplished, according to the invention, by means of a composition that contains at least one lipid transfer protein as well as at least one lipophilic and/or amphiphilic component, as well as water, and furthermore has at least one emulsifying protective colloid that is selected from among natural hydrocolloids, particularly the group comprising caseinates, animal protein derivatives, gelatins, albumins, animal-marine colloids and derivatives thereof, or the group of synthetically/partially synthetically produced colloids or mixtures thereof.

In this way, a stable emulsion system is formed, on the one hand, in which the transfer proteins as such develop their desired catalytic effect, without intervening in the emulsifying system. On the other hand, protective colloids are used, which not only guarantee a stable emulsion, e.g. as a cream, but also make an emulsifying effect of the transfer proteins superfluous. This would be contraindicated in the case of the intended purpose, since otherwise the structures that are supposed to be transferred, namely the lipid components, would be emulsified. Using the special protective colloids, it was therefore surprisingly possible to allow an improved lipid transfer, causing the lipid metabolism of the cells, particularly of the skin, to be balanced out, for one thing, and for another, to be better supplied with lipid active substances. In this connection, it is possible to do without traditional O/W (=hydrophilic) emulsifiers.

Thus the combination according to the invention, i.e. the system of lipophilic and/or amphiphilic components, lipid transfer proteins, and protective colloids is particularly suitable for broad areas of application, particularly as a cosmetic or dermatological agent that can be applied externally/topically, also using additives. The composition can be present, in this regard, as a cream, lotion, milk, e.g. in the form of a W/O, O/W, particularly an O/W composition, ointment, gel, spray, or the like, depending on the proportion of the individual components or the water content, respectively. In particular, the composition can be used for the production of agents for influencing lipid cell structures, particularly for cleansing, care, sun protection and/or preventive or therapeutic treatment of the skin of mammals, particularly for improving the skin cell structure of aging, damaged, particularly light-damaged, dry, sensitive, inflamed skin, for protecting mammals against sun, and also as a shower preparation, care cream, or lotion.

The compositions according to the invention can also be applied to mucous membranes, whereby a particularly good lipid transfer is also possible. The amount information relates to wt.-% unless otherwise indicated.

Particularly suitable compositions comprise proteins from animal, plant, marine microbiological sources such as grains, tubers, milk, silk, particularly animal sources as described in the following.

The lipids are selected, in particular, from among lecithins, sphinglipids, cholesterols, phospholipids, sphingolipids, gangliosides, cerebrosides, ceramides, lipophilic or amphiphilic plant substances, glycerides, hydrocarbons, natural or synthetic fats, oils, waxes, fatty alcohols, fatty acid esters, fatty acid partial esters, silicone oils, silicone waxes, or mixtures thereof, particularly from combinations of at least one substance from the group of the more strongly polar lipids/amphiphilics in combination with at least one substance from the group of the more weakly polar lipids/amphiphilics, as described below.

Preferably, the composition contains water, e.g. 0.1 to 85%, particularly 10 to 60%. In another embodiment, 35 to 70% water, above all, can also be present.

### MORE DETAILED DESCRIPTION OF THE INGREDIENTS

1) Lipid Transfer Proteins

The lipid transfer proteins according to the invention are preferably selected from among animal, plant, microbial, and marine proteins. They can also be obtained synthetically or by means of gene technology.

These proteins therefore have an average molecular mass between 2,000 and 40,000 D, particularly 2,000 to 30,000 D, above all 2,000 to 15,000 D.

The suitable proteins particularly include those from grains having an average molecular mass of 4,000 to 10,000 D, proteins from tubers or fruits having an average molecular mass of 5,000 to 20,000 D, proteins from milk having an average molecular mass of 2,000 to 8,000 D, proteins from silk, microorganisms, marine sources having an average molecular mass of 10,000 D, above all 15,000 D to 40,000 D, or mixtures thereof.

Suitable tubers are potatoes, further suitable fruits are ylang ylang, Jerusalem artichoke, or the like. Marine sources are, for example, algae, mussels, clams and other seafood. Suitable microorganisms are yeasts, bacteria.

Lipid transfer proteins that are particularly preferred are those from grains, above all those that can be obtained from wheat, barley, corn and, above all, also from oats.

Furthermore, animal proteins from milk as well as corresponding hydrolysates (average molecular mass above all 2,000 to 4,000 D) are advantageously suitable. Silk proteins are preferably selected from among silkworm protein and spun silk protein.

Very particularly preferably, proteins of the stated type having an average molecular mass of 2,000 to 30,000 D, particularly 2,000 to 15,000 D, are chosen. Among these, grain proteins and, in particular, milk proteins and also silk proteins are particularly suitable.

Also, mixtures of lipid transfer proteins such as mixtures of plant proteins and animal proteins, or of marine products with plant and/or animal proteins, are particularly suitable.

The proteins can be obtained in known manner, by means of slurrying them up in water, centrifugation, and chromatography, whereby other treatment methods such as homogenization, de-greasing with organic solvents, precipitation, dialysis, filtration, which are known to a person skilled in the art, can be used, see the aforementioned U.S. Pat. No. 5,776,470. Other methods for obtaining proteins of this type are described, for example, in EP-A 0 620 979 and the literature cited therein.

In this manner, proteins or hydrolysates thereof, having the stated molecular masses, are obtained, which are then used in the compositions according to the invention. They particularly demonstrate catalytic transfer activity, which can be determined according to the known resonance energy transfer test (RET test), see Nichols, J. W., Pagano, R. E., J. Biol. Chem. 258 (1983), 5368-5371. According to the latter, two liposome species having different lipid composition can be used, for example, whereby one is used as a probe and the resonance energy transfer system contains with two fluorophores that are in resonance with one another, such as nitrobenzoxadiazole and rhodamine B, for example. In the case of spatial proximity of the two fluorophores in the probe lipisomes, the inherent fluorescence of the excited fluorophore disappears, since the energy is directly absorbed by the second fluorophore. If the distance between the two fluorophores is now changed by means of protein-catalytic lipid transfer, the inherent fluorescence of the excited fluorophore occurs once again. It is a direct measure of the lipid transfer.

In particular, the proteins used according to the invention are at least dispersible in water, but above all they are soluble in water. They act, above all, as a transfer vehicle. They can be used in amounts of 0.01 to 10 wt.-%, above all 0.01 to 8 wt.-%.

Preferably, amounts of 0.01 to 5 wt.-%, particularly 0.01 to 2 wt.-%, above all 0.01 to 0.95 wt.-%, and very particularly 0.01 to 0.85 or also 0.1 to 0.85, particularly 0.1 to 0.5 wt.-% are chosen.

If combinations are used, particularly of animal proteins and plant proteins of the type stated, this can take place in a ratio of 10:1 to 1:10, particularly 5:1 to 1:1, for example. Milk protein is particularly suitable, particularly hydrolyzed milk protein in combination with plant protein (e.g. oat protein or a hydrolysate thereof, or marine proteins.

2. Lipid Components

Both more strongly polar substances and more weakly polar substances are suitable as lipid components, namely lipophilic or amphiphilic components.

Thus, natural or synthetic (saturated, unsaturated) fats/oils or waxes, above all from the group of the more weakly polar substances, can be used. These include natural, partially synthetic, or synthetic oils, hydrocarbons such as squalene, squalane, particularly also liquid paraffins, isoparaffins, dioctyl cyclohexanes, isodecane, isohexadecanes.

Also, fatty acids, e.g. C2-18 alcohol fatty acid esters such as isopropyl fatty acid esters (palmitate, myristate, isostearate, oleate), decyl oleates, hexyl laureates, C 12-15 alkyl benzoates, dicaprylyl carbonates, as well as branched fatty acid esters such as cetearyl octanoates, di-n-butyl adipates are suitable;

Furthermore, C₈₋₂₂ fatty alcohol ethers such as dicaprylyl ethers, or fatty alcohol esters such as C₁₂₋₁₃ alkyl lactates, particularly glycerides (C₁₂₋₂₂ fatty acid glycerides) such as triglycerides, particularly medium-chain (neutral oils) such as caprylic/capric triglycerides as well as their polyol esters such as propylene glycol dicaprylates/dicaprates are suitable;

Also, natural (saturated/unsaturated) fats and oils are used, such as sunflower oil, soybean oil, peach pit oil, apricot pit oil, grape seed oil, castor bean oil, peanut oil, almond oil, mink oil, wheat germ oil, sesame oil, thistle oil, almond oil, avocado oil, shea butter or illipe butter; natural liquid waxes, e.g. jojoba oil or its substitute, oleyl erucate;

Furthermore, silicone derivatives such as silicone oils and waxes, e.g. polydimethyl siloxanes such as dimethicone, cyclomethyl siloxanes such as cyclopentasiloxanes, phenyl methyl siloxanes, such as phenyl dimethicone, or alkyl-polymethyl siloxane copolymers such as cetyl dimethicone, stearyl dimethicone, dialkoxy dimethyl polisiloxanes such as stearoxy dimethicone, behenoxy dimethicone;

Also, waxes such as natural or bleached bee's wax (C₂₀₋₄₀ alkyl stearate) or micro wax or hydrocarbon waxes such as mineral wax, as well as hydrogenated castor beam oil or synthetic waxes such as cetyl palmitate or myristyl myristate, or stearyl stearate, are suitable.

C₈₋₂₂ fatty alcohols such as oleyl alcohol, octyl dodecanol, cetyl/stearyl alcohol, are suitable as more strongly polar lipid components;

Also, C₁₂₋₂₂ fatty acid partial esters of multivalent C₂₋₆ alcohols such as, in particular, monoglycerides, or diglycerides, e.g. glycerol monostearate/distearate, mixtures thereof; polyol C₁₂₋₂₂ fatty acid esters such as PEG-7 glyceryl cocoate, propylene glycol dicaprylate/dicaprate, mixtures such as hexyldecanol and hexyldecyl laurate can be used.

Here, the following are also particularly suitable: liposomal lipid components or derivates thereof, as well as, in particular, lecithin, such as soybean lecithin and egg lecithin; phospholipids such as phosphatidyl choline, phosphatidyl serine, phosphatidyl ethanolamine, phosphatidyl glycerol, phosphatidyl inositol, gangliosides, cerebrosides, cholesterol, and ceramides, such as ceramide-2, -3, -6, for example, as well as sphingolipids. Very particularly preferably, the lipid component(s) are selected from among the stated hydrocarbons, (C₈₋₂₂) fatty alcohols, ethers, (C₂₋₆ alcohol) (C₁₂₋₂₂) fatty acid partial esters, (polyol C₁₂₋₂₂)/C₂₋₁₈ fatty acid esters, mono, di/tri (C₁₂₋₂₂) glycerides, natural or synthetic oils or fats, lecithins, phospholipids ceramides, sphingolipids, gangliosides, cerebrosides, cholesterols, silicone oils, silicone waxes, lipophilic or amphiphilic plant substances, or mixtures thereof.

In particular, liquid paraffins, natural fats/oils/waxes, mono/triglycerides of C₁₂₋₂₂ fatty acids, C₁₂₋₂₂ fatty alcohols, C₁₂₋₂₂ fatty acid partial esters of C₂₋₆ multivalent alcohols, polyol C₁₂₋₂₂ fatty acid ester lecithins, phospholipids, among them, above all, those mentioned above, gangliosides, cerebrosides, cholesterol, ceramides, sphingolipids, polysiloxane compounds, lipophilic or amphiphilic vitamins, and mixtures thereof are suitable. It has furthermore been shown that a particularly advantageous embodiment of compositions according to the invention is obtained if the lipophilic/amphiphilic substances represent a combination of one or more more strongly polar substances and one or more more weakly polar substances. The more weakly polar lipophilic substances include hydrocarbons, natural or synthetic fats or oils or waxes, triglycerides, silicone derivatives as described, in particular polysiloxane compounds. The more strongly polar substances include lecithins, ceramides, cholesterols, phospholipids, sphingolipids, gangliosides, cerebrosides, lipophilic or amphiphilic plant substances, mono/diglycerides of C₁₂₋₂₂ fatty acids, C₁₂₋₂₂ fatty alcohols, C₁₂₋₂₂ fatty acid partial esters or multivalent C₂₋₆ alcohols, C₂₋₆ polyol C₁₂₋₂₂ fatty acid esters.

If one or more representatives of the more strongly polar group and one or more representatives of the more weakly polar group are combined, in each instance, this unexpectedly results in an even better lipid transfer, together with the lipid transfer protein(s), possibly on the basis of an adduct formation.

In another preferred embodiment, these lipids or lipid combinations are used together with the transfer proteins having an average molar mass of 2,000 to 30,000 D, particularly those selected from animal or animal and plant transfer proteins.

Combinations of liposomal lipid components, mono/triglycerides of C₁₂₋₂₂ fatty acids, C₁₂₋₂₂ fatty alcohols, and fatty acid esters/partial esters as described, vitamins, and hydrocarbons, are very particularly preferred.

Also, silicone derivatives as described, particularly polysiloxanes, above all in combination with the combinations stated above, can be used in very particularly preferred manner.

The lipophilic/amphiphilic component(s) are present in amounts of 0.1 to 40 wt.-%, preferably 1 to 30, and very particularly 1 to 20 wt.-%.

3. Protective Colloids

The protective colloid(s) used according to the invention are those having a natural origin, namely animal or animal-marine origin, and derivatives thereof, or synthetically or (partially) synthetically produced colloids, particularly hydrocolloids. These are, in particular, neutral non-ionic or ionic. Those having a natural origin, particularly an animal origin, are particularly preferred. Furthermore, combinations of representatives of these groups (e.g. animal/synthetic and/or partially synthetic or several from one group) are very well suited to act as emulsifiers both individually and quasi in the composition.

In this regard, the following are particularly suitable, particularly from the group of natural representatives: (animal) caseinates, protein derivatives of an animal nature, gelatins, albumins, or mixtures thereof.

Hydrolysates having a higher average molar mass are suitable as protein derivatives of an animal nature (or accordingly synthetically produced products).

Water-soluble casein compounds, particularly salts of casein, such as sodium caseinate, are very particularly preferred as protective colloids (having a natural, animal origin). The relative molar masses can lie, in particular, in the range of 15,000 to 30,000 D.

Types A and B according to Ph. Eur. 4 are suitable as gelatins. Also suitable are albumins from egg, serum, which can be obtained from the natural starting material by means of chromatography.

The stated compounds are known as such.

The synthetic/partially synthetic colloids preferably include water-soluble (poly) (meth)acrylate polymers such as carbomers, see Ph. Eur. 4, or water-soluble cellulose derivatives such as methylcellulose, hydroxypropyl cellulose, hydroxyethyl cellulose, hydroxypropyl methylcellulose.

These last colloids mentioned particularly have an average molar mass of more than 40,000 D, e.g. carbomers 1.25 to 4 million.

The protective colloid(s) are preferably used in amounts of 0.01 to 10 wt.-%, above all 0.01 to 5 wt.-%, and above all 0.1 to 4 wt.-%.

In another embodiment, they can also be present in amounts of 0.1 to 3 wt.-%.

The compositions according to the invention preferably contain not only these three stated components, lipid/transfer protein/protective colloid, but also water, whereby the amount of the latter depends on the galenic formulation and in this regard can lie between 0 and 80%, particularly 5 and 75%, furthermore also 35 to 65 wt.-%. In this, additives, e.g. 0.01 to 40 wt.-%, can preferably also be present.

Particularly preferably, the compositions are therefore produced in the form of O/W-like emulsions such as creams, but any other type of formulation is possible, such as gel, ointment, spray, or also W/o emulsion.

Compositions that contain 0.01 to 10 wt.-%, particularly 0.01 to 3 wt.-%, above all, however, 0.1 to 0.9 wt.-% of one or more lipid transfer proteins, 0.01 to 40 wt.-%, preferably 0.1 to 25 wt.-% of one or more lipophilic and/or amphiphilic components, particularly 1 to 20 wt.-%, furthermore 0.01 to 10 wt.-%, particularly 0.1 to 6 wt.-% of one or more protective colloids, as well as 0.01 to 30, preferably 0.1 to 20 wt.-% additives and, as the remainder, water are particularly preferred. The lipid combinations mentioned, consisting of more strongly polar and more weakly polar substances in combination with the transfer proteins having an average molar mass of 2,000 to 30,000 D are very particularly preferred.

4. Additives

The compositions according to the invention can contain additives in an amount of 0 to 40%, particularly 0.01 to 40 wt.-%, preferably 0.1 to 30 wt.-%, above all 0.1 to 20 wt.-%, as explained.

The preferred additives include active substances, conditioning agents, preservatives, dyes, perfume substances, care substances. These can be present, in particular, in amounts of 0.01 to 10%, in each instance, preferably 0.01 to 5%.

The active substances can be soluble in water or fat, and are particularly selected from the extracts of plants, synthetically produced substances corresponding to these extracts, and analogous derivatives thereof, vitamins, as well as substances that influence the skin, and mixtures thereof.

Extracts of plants are, for example, those of ylang ylang, pine needles, cypress, thyme, mint, limes, oranges, grapefruit, mandarin oranges, juniper, valerian, lemon balm, eucalyptus, thyme, palmarosa, rosemary, lavender, rosewood, lemon grass, spruce needles, pine needles, ginger extract, currant extract, linden blossom extract, calendula extract, magnolia extract, algae extract, aloe vera extract, pineapple extract, guava extract, echinacea extract, ivy leaf extract, birch leaf extract, calendula extract, hibiscus extract, burdock root extract, witch hazel extract, hydrocotylidis herba extract, quince extract, water lily extract, cinnamon extract, or mixtures thereof.

Suitable vitamins are, for example, Vitamins A, B, E, C, or suitable derivatives thereof, such as esters, e.g. palmitate, acetate, or phosphate.

Other suitable active substances are minerals and trace elements such as copper, zinc, magnesium, or their derivatives, particularly salts, such as Zincidone® (zinc PCA), zinc gluconate, or copper gluconate.

Furthermore, astringent and sebum-regulating substances can be used, such as hydroxydecanoic acid, pyridoxine, niacinamide, glycerin, caproyl collagen amino acids, sebacic acid, cinnamonum zeylanicum.

Also, cooling/soothing active substances such as menthyl lactate or sodium hyaluronate, wheat germ extract, saccharomyces cerevisiae extract can be incorporated.

Furthermore, active substances that promote blood circulation, such as nicotinic acid derivatives such as methyl nicotinate or tocopheryl nicotinate, alphahydroxy and betahydroxy acids and their derivatives, e.g. glycolic acid, malic acid, citric acid, tartaric acid, lactic acid, salicylic acid, isopropylbenzyl salicylates, C12-13 alkyl lactate minoxidil or also antiphlogistic and antibacterial substances such as triterpenes, e.g. ursolic acid, glycyrrhizinic acid or glycyrrhetinic acid, and their derivatives, e.g. stearyl glycyrrhetinate, potassium glycyrrhinate; pantothenic acid derivatives, e.g. D-panthenol, panthenyl triacetate; allatoin; bisabolol; azulenes, e.g. chamazulene or guajazulene; phytosphingosines; triclosan; chlorhexidine derivatives can be used.

Furthermore, substances having an antioxidant effect or a cell-protection effect, such as polyphenols, flavonoids, e.g. rutin, ferulaic acid, and their esters, or isoflavones such as soybean isoflavones or co-enzyme Q 10, hyaluronic acid, oleanolic acid, and/or ursolic acid can also be used as active additives.

In contrast, strongly oxidative substances such as hydrogen peroxide, in particular, are not used.

Other active substances are UV filters such as UVB, WVA, and broadband filters, as they are generally known, e.g. cinnamic acid esters, salicylates, analogous known substances, or also inorganic UV filters such as zinc oxide and titanium dioxide, particularly micronized and/or coated.

Other active substances that can be used are biotin, allantoin, panthenol, niacinamide, urea, inositol. Depending on the purpose of use, active substances that are soluble in oil or water can also be used or combined for cosmetic and/or dermatological/pharmaceutical purposes.

Dermatologically/pharmaceutically active substances can be, in particular, antimicrobially active substances (antibiotics; antimycotics, antiviral substances), hormones or hormonally active substances such as sex hormones, wound cleansing agents, analgesics, cytokins, cytostatics, anti-acne agents, immune suppressives, pigmentation regulators, agents against photodermatoses, or the vasoactive/antiphlogistic substances named above, or combinations thereof.

Other additives include, as mentioned, usual conditioning agents in suitable amounts, such as acrylamides, starch, such as methyl cellulose, hydroxyethyl cellulose, carmellose-Na, hydroxypropyl cellulose, hypromellose, or starch glycols, rice starch, wheat starch, corn starch, and potato starch, for example, furthermore also hydrophobically modified starches (corn starch modified), aluminum starch octenyl succinate, hydroxypropyl starch phosphate ester, or mixtures of the latter such as aluminum starch octenyl succinate, acrylates copolymer, magnesium carbonate, and the like are suitable.

Furthermore, acrylates as well as polysaccharides (e.g. xanthan gum), cellulose derivatives such as hydroxypropyl methylcellulose or ethyl cellulose, or silicates such as magnesium aluminum silicate and the like should be mentioned here.

Known acrylamides are polyacrylamide, or mixtures containing polyacrylamide. Carboxy-vinyl mixed polymerizates having a high molecular weight (1-3 million) as well as their copolymers can be used as acrylates, particularly after neutralization by means of alkali, such as carbomer, or the already neutralized carbomers such as sodium carbomer. Also, acrylate copolymers can be used, e.g. arylates/C10-30 alkyl acrylate cross-polymer, or acrylamide alkyl sulfonic acid derivatives such as ammonium acryloyl dimethyl taurate/VP copolymer.

Furthermore, complexing agents such as EDTA, Na salt, means for adjusting pH, e.g. citric acid, soda lye, solvents such as propylene glycol or alcohols, salts such as NaCl, starch or starch derivatives, or mixtures thereof, can be contained as conditioning agents.

Other conditioning agents are, in particular, monoalkyl and dialkyl phosphates or glyceryl stearate.

Particularly suitable care substances are phytosterols (essentially mixtures of β-sitosterol, campesterol, and stigmasterol or ethoxylated derivatives thereof), such as those from canola oil. Also lecithin (e.g. soybean lecithin), phosphatidyl choline, phosphatidyl serine or diethanolamine, or mixtures thereof with the aforementioned phytosterols can be used here.

Furthermore, silicone oils such as dimethicones or silicone (co)polymers, e.g. di-cyclomethicone or divinyl dimethicone/dimethicone copolymers, C12-13 pareth-3/C12-13 pareth-23) can be used here.

Other care substances are moisturizing agents such as glycerin, propylene glycol, or polyethylene glycols, propylene glycol, butylene glycol, sorbitol, or polymers, e.g. polyquaternium types such as polyquaternium 39, collagen or its hydrolysates, amino acids, urea, polysaccharide (biosaccharide gum 1), glucosaminoglycanes, e.g. hyaluronic acid or sulfated glucosaminoglycanes such as chondroitin sulfate, dermatane sulfate, keratane sulfate, heparane sulfate, particularly their Na salts or heparin-Na, glucanes, e.g. β-glucane, e.g. oat β-glucane, annanes such as konjac mannane, or commercially available moisturizing agents such as inositol), salts, e.g. sodium lactate, DL-2-pyrrolidone-5-carboxylic acid, Na salt.

Polyethylene/propylene glycol or as well as polysaccharide compounds, algae extracts, and/or hyaluronic acid, Na salt are preferred.

Other possible additives are suitable surfactants such as sarcosinates, acyl glutamates, isethionates, polyglycosides.

In addition, one or more preservatives and can be present, if necessary. Suitable preservatives are, for example, iodopropynyl butyl carbamate, DMDM hydantoin, phenoxyethanol and other commonly used preservatives, such as sorbinic acid and dehydracetic acid, and their salts, methyl dibromoglutarononitrile, etc., or combinations thereof, or other acids such as benzoic acid or salicylic acid, or benzyl alcohol or esters such as p-hydroxy benzoic acid esters, e.g. methyl paraben, ethyl paraben, propyl paraben, butyl paraben, isobutyl paraben, preferably methyl paraben or propyl paraben or mixtures thereof, or climbazol or suitable combinations of the stated substances, such as methyl paraben, propyl paraben, and sorbinic acid are possible. Mixtures, e.g. of DMDM hydantoin and iodopropynyl butyl carbamate or of phenoxy ethanol with p-hydroxy benzoic acid ester, isothiazolinone, methyl dibromoglutanonitrile, etc., are particularly suitable.

Suitable perfume substances (e.g. preferably 0.01 to 5%) are, for example, the ether oils named under active substances, or commercially available perfume mixtures.

Preferably, the components known for products of this type are chosen as dyes (e.g. preferably 0.01 to 2%).

The type and amount of the additives is based on the desired form of use and the desired purpose. It is preferred if at most 30%, preferably 0.01 to 25%, above all also 0.1 to 25%, or 1 to 20%, particularly 3 to 20% of additives are present.

Particularly preferably, 0.01 to 25% additives, which are, above all, selected from among active substances, care substances, conditioning agents, perfume substances, are suitable.

Above all, the stated vitamins, particularly tocopherol or derivatives thereof and/or panthenol are also preferably used, particularly also in combination with ursolic acid and/or oleanolic acid and/or hyaluronic acid (e.g. sodium salt) and/or Q 10.

Use

Depending on the intended purpose, the compositions according to the invention can also be used, by means of selecting suitable additives, for cosmetic and dermatological/therapeutic treatment of the skin, hair, or mucous membranes. The compositions can be present, in this regard, as a cream, lotion, milk, ointment, gels, sprays, and used as such for care, cleansing (e.g. shower, mask, cream). Use in the form of a cream is particularly preferred. By means of the combination according to the invention, the transfer proteins used can develop their effect as a vehicle for lipophilic/amphiphilic components. By means of the transfer of lipids, the renewal of the cellular and extracellular lipid structures of the cells, particularly of the skin, takes place. In this way, a good transfer rate for active substance transport can be achieved, without any complicated methods being required for this, such as in the case of liposomes, for example. Thus, not only is it possible to maintain intact, not rigidly crosslinked lipid structures, as they occur particularly as part of aging, but also an improved treatment of skin function problems is possible, since a rapid elimination of the problem can be brought about by means of a targeted transport of a lipid component that is required, in each instance. Vice versa, undesirable lipid substances can be removed by means of this system. Thus, for example, not only is it possible to undertake general cosmetic use for cleansing, care, symptoms of aging skin, but also therapeutic use, for example in the case of inflamed conditions, irritated conditions of the skin, e.g. as the result of allergies or irritations. In particular, it is possible to use the compositions according to the invention, when used topically externally, to influence skin cell structures of aging, damaged, particularly light-damaged, dry, sensitive, inflamed skin of mammals, particularly humans.

Production of the Products

The compositions according to the invention are produced in that the water-soluble or water-dispersible components are worked into water, the lipid phase is prepared analogously, and both phases are mixed at suitable temperatures, such as 20 to 80° C., for example, preferably 30 to 60° C. to room temperature, in such a manner that a stable emulsion is obtained, and any phases that are temperature-unstable at room temperature are added while stirring, if necessary. Emulsification takes place in such a manner that on the one hand, a high dispersity of the coherent phase is achieved, and on the other hand, depolymerization of the protective colloids, for example because of overly high shear forces, is prevented.

### EXAMPLES

The invention will be explained in greater detail using the following examples.

### Example 1

### O/W Emulsion

| | | % |
|---|---|---|
| Item | INCI | [w/w] |
| Fatty phase | | |
| 1 | Stearyl alcohol | 2.0 |
| 2 | Glyceryl stearate | 2.0 |
| 3 | Isopropyl stearate | 6.0 |
| 4 | Persea gratissima (avocado | 3.0 |
| | oil) | |
| 5 | Dicaprylyl ether | 3.0 |
| 6 | Squalane | 5.0 |
| 7 | Tocopheryl, hydrogenated palm | 0.05 |
| | Glycerides citrate | |
| Water phase | | |
| 8 | Water | To 100 |
| 9 | Glycerin | 2.0 |
| 10 | Butylene glycol | 2.0 |
| 11 | Disodium EDTA | 0.1 |
| 12 | Sodium caseinate | 3.0 |
| 13 | Hydrolyzed milk protein | 0.5 |
| 14 | Avena sativa (oat protein) | 0.5 |
| Active substance | | |
| phase | | |
| 15 | Cyclomethicone | 1.0 |
| 16 | Tocopheryl acetate | 1.0 |
| 17 | Preservatives | q.s. |
| 18 | Perfume | q.s. |

### Example 2

### O/W Emulsion

| Item | INCI | % [w/w] |
|---|---|---|
| 1 | Cetyl alcohol | 2.0 |
| 2 | C12-15 alkyl benzoate | 5.0 |
| 3 | Squalane | 4.0 |
| 4 | Cetearyl isononanoate | 3.0 |
| 5 | Tocopheryl acetate | 4.0 |
| 6 | Acrylates/C10-30 alkyl acrylate cross- | 0.2 |
| | polymer | |
| 7 | Water | To 100 |
| 8 | Hydroxypropylethyl cellulose | 0.2 |
| 9 | Glycerin | 3.0 |
| 10 | Disodium EDTA | 0.1 |
| 11 | Avena sativa (oat protein) | 0.05 |
| 12 | Triethanolamine | 0.25 |
| 13 | Preservative(s) | q.s. |
| 14 | Perfume | q.s. |

### Example 3

### W/O Emulsion

| Item | INCI | % [w/w] |
|---|---|---|
| Phase A | | |
| 1 | Polyglyceryl-4 isostearate, cetyl | 5.0 |
| | PEG/PPG-10/1 dimethicone, | |
| | hexyl laurate | |
| 2 | Cyclodimethicone | 15.0 |
| 3 | C12-15 alkyl benzoate | 5.0 |
| Phase B | | |
| 4 | Sodium chloride | 1.0 |
| 5 | Water | To 100 |
| 6 | Butylene glycol | 3.0 |
| 7 | Ursolic acid | 0.1 |
| 8 | Tocopheryl acetate | 0.5 |
| 9 | Hydrolyzed corn protein | 0.3 |
| 10 | Hydrolyzed milk protein | 0.3 |
| 11 | Preservative(s) | q.s. |
| 12 | Perfume | q.s. |

1. Composition that can be applied externally, containing at least one lipid transfer protein as well as at least one lipophilic and/or amphiphilic component and water, wherein the composition has at least one emulsifying protective colloid, which is selected from among natural hydrocolloids, comprising caseinates, protein derivatives of an animal nature, gelatins, albumins, animal-marine colloids, and derivatives thereof; or synthetically or partially synthetically produced colloids or mixtures thereof.

2. Composition according to claim 1, wherein the lipid transfer protein(s) is/are selected from among animal, plant, microbial and marine proteins or mixtures thereof.

3. Composition according to claim 1, wherein the lipid transfer protein(s) is/are selected from among proteins from grains having an average molecular mass of 4,000 to 10,000 D, tubers or fruits having an average molecular mass of 5,000 to 20,000 D, proteins from milk having an average molecular mass of 2,000 to 8,000 D, proteins from silk, microorganisms, marine sources having an average molecular mass of 10,000 to 40,000, or mixtures thereof.

4. Composition according to claim 1, wherein the lipophilic and/or amphiphilic component(s) is/are selected from lecithins, sphinglipids, cholesterols, phospholipids, sphingolipids, gangliosides, cerebrosides, ceramides, lipophilic or amphiphilic plant substances, glycerides, hydrocarbons, natural or synthetic fats, oils, waxes, fatty alcohols, fatty acid esters, fatty acid partial esters, silicone oils, silicone waxes, or mixtures thereof.

5. Composition according to claim 1, wherein at least one substance from the group of the more strongly polar lipids/amphiphiles in combination with at least one substance from the group of the more weakly polar lipids/amphiphiles are selected as the lipophilic/amphiphilic components.

6. Composition according to claim 1, wherein the lipophilic and/or amphiphilic component(s) is/are selected from one or more substances of the group of hydrocarbons, natural or synthetic fats, oils, waxes, fatty acid esters, fatty alcohol ethers, polysiloxane compounds, triglycerides, in each instance, in combination with one or more substance from the group of lecithins, sphingomyelins, cholesterols, phospholipids, sphingolipids, gangliosides, cerebrosides, lipophilic or amphiphilic plant substances, mono/diglycerides of C₁₂₋₂₂ fatty acids, C₁₂₋₂₂ fatty alcohols, C₁₂₋₂₂ fatty acid partial esters of C₂₋₆ alcohols, polyol-C₁₂₋₂₂ fatty acid esters.

7. Composition according to claim 1, that wherein 0.01 wt.-% to 30 wt.-% additives are contained.

8. Composition according to claim 1, wherein 0.01 to 10 wt.-% of one or more lipid transfer proteins, 0.01 to 40 wt.-% of one or more lipophilic and/or amphiphilic components, 0.01 to 10 wt.-% of one or more protective colloids, 0.01 to 30 wt.-% additives and, as the remainder, water are contained.

9. Composition according to claim 1, wherein the protective colloid is selected from among substances having a natural origin.

10. Composition according to claim 1, wherein it is present in the form of a cream, lotion, milk, ointment, gel, spray.

11. Use of a composition according to claim 1, for the production of agents for influencing lipid cell structures.

12. Use according to claim 11 for cleansing, care, and/or prophylactic or therapeutic treatment of the skin of mammals.

13. Use according to claim 11 for improving the skin cell structure of aging, damaged, particularly light-damaged, dry, sensitive, inflamed skin of mammals.

14. Use of a composition according to claim 1 for cosmetic treatment of the skin.

15. Use according to claim 14, cosmetic treatment is cleaning, care, sun protection.

16. Use of a composition according to claim 14 as a shower preparation, care cream or lotion.

17. Use of a composition according to one claim 1 for therapeutic treatment of the skin of mammals.

18. Use according to claim 17 for improving the skin cell structure of aging, damaged, particularly light-damaged, dry, sensitive, inflamed skin of mammals.
